(19) United States
(12) Patent Application Publication
(10) Pub. No.: US 2022/0401129 A1
(43) Pub. Date: Dec. 22, 2022

(54) CATHETER FOR THE TRANSFER OF HUMAN EMBRYOS WITH SECTION OF TRANSVERSE FOLDS AND ERGONOMIC HANDLE

(71) Applicant: Eleftherios Meridis, Athens (GR)

(72) Inventor: Eleftherios Meridis, Athens (GR)

(21) Appl. No.: 17/428,474

(22) PCT Filed: Nov. 23, 2020
(86) PCT No.: PCT/GR2020/000061

§ 371 Date: Aug. 4, 2021

(30) Foreign Application Priority Data
Mar. 3, 2020 (GR) 20200100117

(51) Int. Cl. A61B 17/435; A61M 25/00

(52) U.S. Cl. CPC A61B 17/435; A61M 25/0054; A61M 2210/1433; A61M 2025/0004

(57) ABSTRACT

A Catheter for the transfer of human embryos including an outer catheter characterized by an integrated element of transverse folds in the shape of an “accordion” at a specific distance from its tip. When being compact, the element of transverse folds is 1 cm long while the distance of its center from the tip of the catheter is 5.5 cm but can vary +/−1 cm. The element of transverse folds has the ability to bend, expand, compress and generally be configured in the desired shape and angle by the operating clinician in order to facilitate its passage through the cervical channel. The shape and angle are capable of being re-configured by the operating clinician if deemed necessary. The element of transverse folds is also capable of automatically adjusting in shape and angle during its passage through the cervix, following the anatomy of the cervical channel.

### FIELD OF THE INVENTION

The present invention refers to a catheter for the transfer of embryos in the intrauterine cavity of a woman, an act that completes every in-vitro fertilization (IVF) treatment cycle for subfertile couples.

### BACKGROUND

These catheters are familiar. The known catheters are consisted of two parts: an outer catheter (guide) made of semi-rigid synthetic material, having a diameter of 5.5-6.6 fr and length of 16.7-17.3 cm and a lumen through which the inner catheter (soft) is introduced. The inner catheter is made of soft synthetic material, with a diameter of 2.8 fr and length of 23-24 cm and has also a lumen.

The inner catheter is the one which is used for “loading” of the embryos that are about to be transferred in the intrauterine cavity and is inserted inside the outer catheter. The outer catheter is supporting the inner catheter due to being more rigid and assists it firstly in entering the cervical channel through the outer cervical os and secondly in performing the necessary maneuvers so that inner catheter passes through the entire cervical channel and reaches the desired point inside the intrauterine cavity.

It becomes evident that an effective design of outer catheter needs to serve two functions: firstly support of the softer inner catheter since it will bend on its own when hold in hand, and secondly assistance of inner catheter to approach and enter the outer cervical os and cervical channel, and to perform the necessary maneuvers by the doctor/clinician so that the inner catheter is inserted deeper inside the cervical channel and the intrauterine cavity until the point where the embryo transfer will take place.

At this point is worth emphasizing on that the faster, smoother and as atraumatic as possible approach of the point inside the intrauterine cavity that the embryo transfer will take place, is decisive for the success of the whole IVF treatment cycle and achievement of embryo implantation and pregnancy. Any difficulty, delay, injury or micro-bleeding during the passage of the inner catheter through the cervical channel can be catastrophic to the implantation process of the embryos.

However the existing catheters in the market have a disadvantage in achieving the above goal due to them being semi-rigid and straight in shape, the result being that on many occasions they approach the outer cervical os at an angle (especially in cases where the cervix protrudes obliquely in the vagina). Furthermore, the cervical channel is not shaped as a straight line but as a curve or sometimes even as the letter “S”. This results to the fact that a standard outer catheter being straight is unable to follow the shape and maneuver appropriately according to the shape of the cervical channel.

Many clinicians, in their attempt to navigate through the “S” shaped cervical channel with the existing catheters, bend with their fingers the end part of the outer catheter, aiming to give it a more anatomical design, that can deal with the curves of the cervical channel. Even so though, the catheter remains stable at the angle given to it by the doctor, resulting in it being unable to manage effectively the angles and curves of the cervical channel. In addition to that, the whole process becomes more difficult due to the fact that the catheter's handle is not ergonomically designed and doesn't have any reference points.

### SUMMARY

This innovation refers to a catheter for the transfer of human embryos. These catheters should have the ability to adjust either automatically or by the operating clinician to the desired shape and angle, so that their entry and passage inside the outer cervical os, the cervical channel and the intrauterine cavity is facilitated.

The proposed catheter has an integrated element of transverse folds in the shape of an “accordion”, which is able to be configured in a way to facilitate the passage of the catheter inside the cervical channel and intrauterine cavity. The proposed catheter has the following advantages. Firstly, it is flexible at a specific section, so that it can automatically and on its own manage the shape of the cervical channel and navigate through it. Secondly, it can be configured to and maintain the desired shape and angle given to it by the operating clinician, in order to achieve an easier, faster and without micro-injuries entry and passage inside the cervical channel and intrauterine cavity. Thirdly, it makes possible the re-configuration of its shape and angle by the operating clinician if deemed necessary. Finally, the configuration at the section of transverse folds is achieved without narrowing the lumen of catheter (α) as to affect the functionality of catheter (β).

### BRIEF DESCRIPTION OF THE DRAWINGS

FIG. 1-3. The proposed embryo-transfer catheter, when catheters (α) and (β) are assembled. More specifically:

FIG. 1. Front View.

FIG. 2. Side View.

FIG. 3. Section.

FIG. 4-6. Detail of the section of transverse folds (1) of catheter (α). More specifically:

FIG. 4. The section of transverse folds in expansion.

FIG. 5. The section of transverse folds in compression.

FIG. 6. The transverse folds in section.

FIG. 7-9. Detail of the ergonomic handle of the catheter, comprised of handles (3,4) of the respective catheters (α) and (β) when in assembly. More specifically:

FIG. 7. Front View.

FIG. 8. Side View.

FIG. 9. Section.

FIG. 10-12. The outer catheter (α). More specifically:

FIG. 10. Front View.

FIG. 11. Side View.

FIG. 12. Section.

FIG. 13-15. The inner catheter (β). More specifically:

FIG. 13. Front View.

FIG. 14. Side View.

FIG. 15. Section.

### LEGEND

1. The transverse folds of the outer catheter (α) as described in the claim.
2. Indicative dots at 1, 2, 3, 4 and 5 cm from the tip of the catheter (α).
3. The handle of outer catheter (α), inside which catheter (β) enters.
4. The handle of inner catheter (β), which connects firmly to the handle of outer catheter (a), thus forming the ergonomic handle.

### DETAILED DESCRIPTION

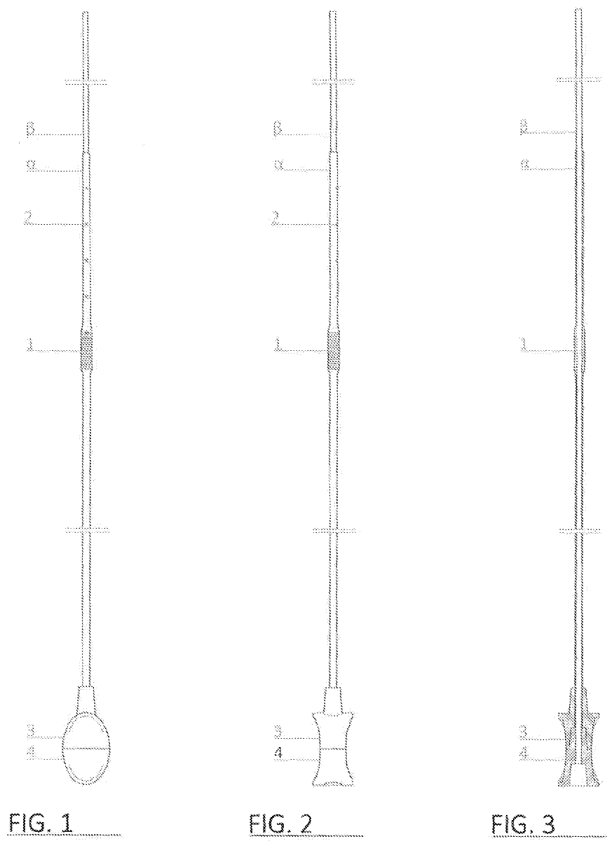

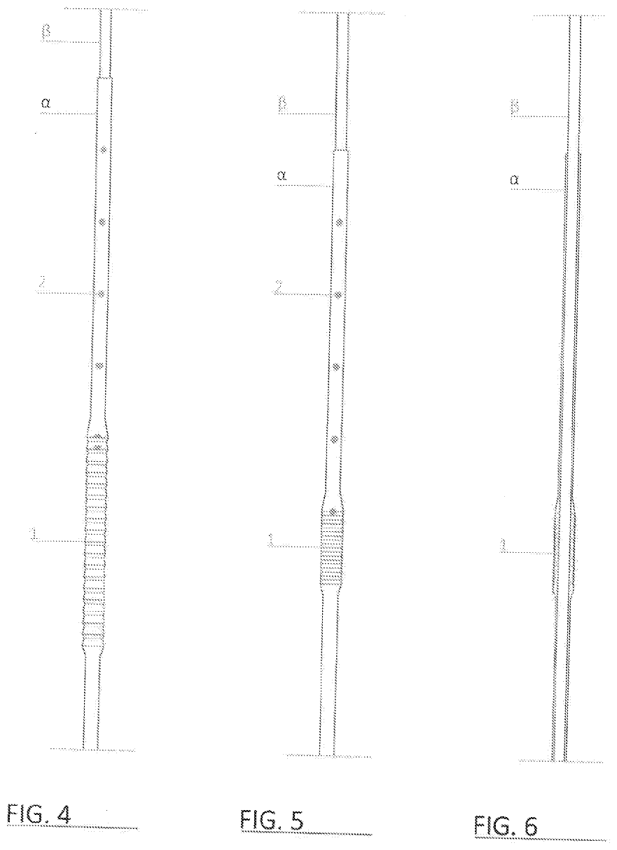

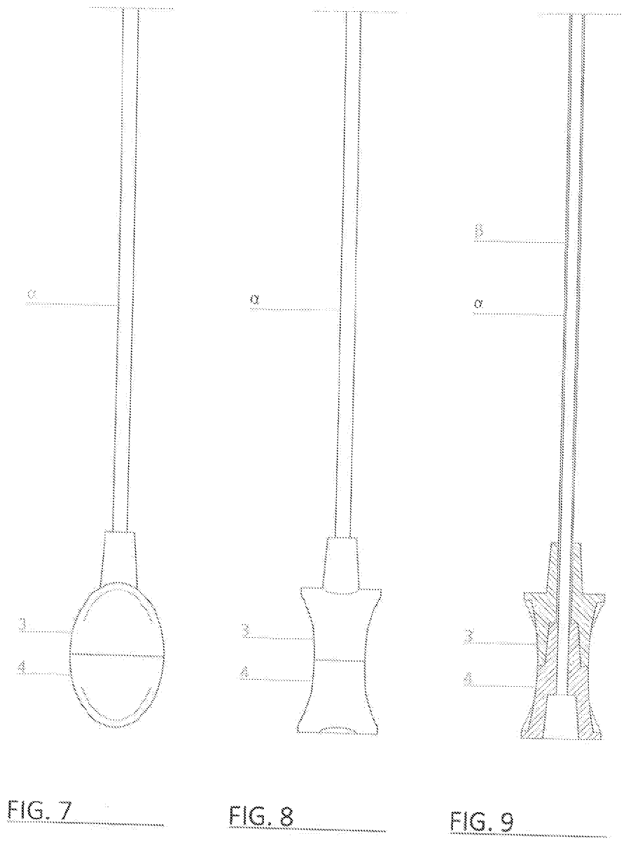

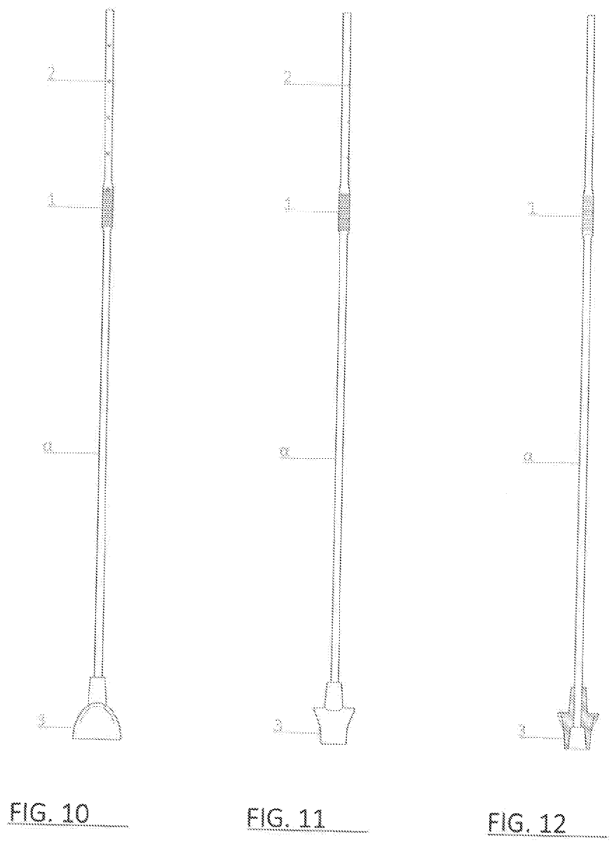

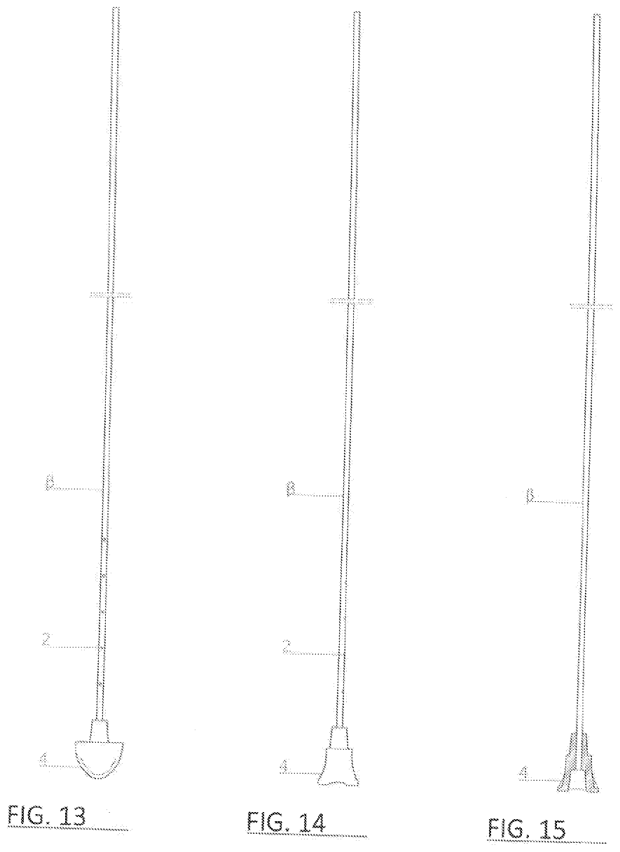

Referring to FIGS. 1-6 and 10-15, the proposed catheter consists of an outer, harder catheter 18.5 cm long (α) and an inner soft catheter 24.5 cm long (β). At a distance between 5 and 6 cm from its tip, catheter (α) has an element of transverse folds (1) in the shape of an “accordion”. The distance of the center of the folds is proposed at 5.5 cm and it can vary +/−1 cm in variations of the same proposal. These folds can expand, or compress, bend and finally be configured in the desired angle without narrowing the lumen of catheter (α) and affecting the functionality of catheter (β). At a distance of 1, 2, 3, 4 and 5 cm from the tip of catheter (α) there are indicative dots (2) that can assist the clinician know, how far has the catheter been inserted inside the cervical channel. The catheter (β) is consistent in configuration, soft, flexible and with a diameter that allows it to be inserted in the lumen of catheter (α). It too has a lumen that is used for the loading, carriage and transfer of the embryos.

Referring to FIGS. 7-9, the base/handle of catheter (α) internally is shaped as a crucible (3), so that it facilitates the entry of catheter (β) and subsequent assembly of the bases/handles of the two catheters. The base/handle of catheter (β) is too shaped as a crucible (4) so to connect firmly with the base/handle of catheter (α) When catheters (α) and (β) are assembled, their bases externally form a handle of elliptical shape, 1.3 cm wide, 2 cm high and 1.1 cm thick. The handle presents with increased thickness perimetrically and reduced centrally thus achieving a larger and more comfortable surface for contact with the operator's hand. This characteristic permits a more accurate and steadier assembly and separation of catheters (α) and (β) by rotation of the two bases (3,4) in opposite directions.

The proposed catheter for the transfer of embryos has the following advantages:
1. It can be autonomous flexible at a certain part of its length so that it can, and on its own navigate the shape of the cervical channel while passing through it, without delays or micro-injuries.
2. It can be bent, expanded, compressed, and generally configured in the desired shape and angle by the operating clinician, in order to achieve easier, faster, and without injuries, passage through the cervical channel.
3. Makes possible a further adjustment of its angle and shape in case this is deemed necessary by the operating clinician.
4. The configuration of the catheter at the section of the transverse folds is achieved without narrowing the lumen of the outer catheter as to affect the functionality of the inner catheter.

While the invention has been described with specific embodiments, other alternatives, modifications and variations will be apparent to those skilled in the art. Accordingly, it will be intended to include all such alternatives, modifications, and variations within the spirit and scope of the appended claims.

What is claimed is:

1. A catheter for the transfer of human embryos comprising an outer catheter characterized by an integrated element of transverse folds in the shape of an “accordion” at a specific distance from its tip;
whereby, when being compact, the element of transverse folds is lcm long while the distance of its center from the tip of the catheter is 5.5 cm but can vary +/−1 cm in variations of the same design;
whereby the element of transverse folds has the ability to bend, expand, compress and generally be configured in the desired shape and angle by the operating clinician in order to facilitate its passage through the cervical channel;
whereby the shape and angle are capable of being re-configured by the operating clinician if deemed necessary;
whereby the element of transverse folds is also capable of automatically adjusting in shape and angle during its passage through the cervix, following the anatomy of the cervical channel; and
whereby the configuration of shape and angle is achieved without narrowing the lumen of the outer catheter as to affect the functionality of an inner catheter.
